(19) United States
(12) Patent Application Publication
(10) Pub. No.: US 2019/0247343 A1
(43) Pub. Date: Aug. 15, 2019

(54) USE OF FENOFIBRIC ACID IN THE TREATMENT OF HEPATIC DISEASES

(71) Applicant: GENCOD, Villeneuve Loubet (FR)

(72) Inventors: Claude LARUELLE, Villeneuve Loubet (FR); Ludovic BONNAFOUS, Mouans Sartoux (FR)

(21) Appl. No.: 16/094,183

(22) PCT Filed: Apr. 13, 2017
(86) PCT No.: PCT/EP2017/059025

§ 371 Date: Apr. 29, 2019

(30) Foreign Application Priority Data
Apr. 15, 2016 (FR) 1653378

(51) Int. Cl. A61K 31/192; A61K 9/00; A61K 9/46; A61P 1/16

(52) U.S. Cl. CPC A61K 31/192; A61K 9/006; A61P 1/16; A61K 9/0007; A61K 9/0019; A61K 9/0056

(57) ABSTRACT

A composition comprising as a sole active ingredient fenofibric acid or one of the pharmaceutically acceptable salts thereof for use in the treatment of liver diseases. The field relates to the treatment and prevention of liver diseases, such as non-alcoholic fatty liver disease (NAFLD), non-alcoholic steatohepatitis (NASH), liver fibrosis or cirrhosis.

### TECHNICAL FIELD

The present invention relates to the treatment and prevention of liver diseases, such as non-alcoholic fatty liver disease (NAFLD), non-alcoholic steatohepatitis (NASH), liver fibrosis or cirrhosis.

The invention relates more particularly to the use of fenofibric acid in the treatment and prevention of NASH.

The liver is the second largest organ in the human body. It fulfils numerous complex functions including: i) defending against diseases and infections, ii) excreting toxins from the body (poisons), such as alcohol, iii) controlling cholesterol levels, iv) aiding blood coagulation, v) releasing bile which breaks down fat and aids digestion, and vi) metabolising foreign bodies and particularly medicinal products administered per os.

Liver diseases do not generally cause obvious signs or symptoms until they are at an advanced stage and the liver is damaged. They may be hereditary (genetic), or caused by a variety of factors that damage the liver, such as viruses and excessive alcohol consumption. Obesity is also associated with liver lesions. Over time, the damage may induce scarification of the liver, which may give rise to potentially life-threatening liver failure for the patient. The symptoms of liver diseases are weakness and fatigue, weight loss, nausea, vomiting and yellowish skin colouring (jaundice).

Non-alcoholic fatty liver disease (NAFLD) is the build-up of fat in hepatocytes which is not due to alcohol consumption. The liver contains a small amount of fat but if more than 5 to 10% of the weight of the liver is fat, it is then referred to as a “fatty” liver: this is known as steatosis. NAFLD tends to develop in subjects who are overweight or obese or who have diabetes, an elevated cholesterol or triglyceride level. Rapid weight loss and poor diet may also cause non-alcoholic fatty liver disease (NAFLD). However, some subjects develop NAFLD even though they have no identifiable risk factors.

Non-alcoholic fatty liver disease NAFLD frequently does not cause any symptoms. If symptoms arise, they may consist of fatigue, weakness, weight loss or loss of appetite, nausea, abdominal pains, yellowing of the skin and eyes (jaundice), itching, build-up of fluid and swelling of the legs (oedema) and abdomen (ascites), and also mental confusion.

The most severe form of non-alcoholic fatty liver disease is known as non-alcoholic steatohepatitis or NASH. Non-alcoholic steatohepatitis causes the liver to swell and become damaged. It is developed by the same at-risk subjects as for NAFLD although, here again, some subjects contract it without having any risk factors.

Most subjects with this non-alcoholic steatohepatitis are between 40 and 60 years of age, and more women than men are affected. Steatohepatitis resembles alcoholic fatty liver disease (AFLD), but occurs in subjects who drink little or generally no alcohol. The progression of NASH can take years. The disease process may stop and, in some cases, regress on its own without specific treatment. On the other hand, the process may also get insidiously worse, causing the onset of scars known as “fibrosis” that build up in the liver. Once the fibrosis increases, cirrhosis develops; the liver becomes seriously damaged, hardened, and incapable of functioning normally.

Non-alcoholic fatty liver disease and steatohepatitis are increasingly frequent, due to a greater number of obese adults. Obesity is also a factor in diabetes and an elevated cholesterol level, which may induce further health complications for someone suffering from NASH.

### STATE OF THE ART

At the present time, there are no medical treatments for non-alcoholic fatty liver disease and non-alcoholic steatohepatitis to date. Only a healthy diet and regular exercise can help prevent or indeed reverse liver damage, in the first stages of the disease.

The causes of the disease, though closely correlated with obesity and excessive sugar consumption are, as frequently in metabolic diseases, multifactorial. Genetic or epigenetic predispositions are evident and some ethnic groups are markedly more exposed than others.

The association of multiple conditions and metabolic disorders such as obesity, diabetes mellitus, cholesterolaemia, lipidaemia, uricaemia, hypertension contribute to the formation of metabolic or cardiometabolic syndrome which is expressed in the liver as NASH.

Non-alcoholic steatohepatitis NASH is a complex disease for which we are only starting to barely understand the delineations and for which both the definition and indices of disease progression (NAS scores) are liable to change with progress in scientific knowledge. It is the result of a metabolic disorder which finds the base thereof in the “fatty” liver.

In the absence of approved and effective treatments, pharmaceutical firms are therefore testing new medicinal products for which the strategies and mechanisms of action are varied. A number of projects are under development, of which a non-exhaustive summary is given hereinafter:

The application WO2015083164A (Galmed Pharmaceuticals) describes pharmaceutical compositions of aramchol salts which are amide conjugates of arachidic acid and 3-aminocholic acid, belonging to the FABAC (fatty-acid/bile-acid conjugates) family. FABACs are known to be effective in lowering blood cholesterol levels and liver lipid levels (steatosis) and also in improving the metabolic parameters associated with “fatty” liver disease and non-alcoholic steatohepatitis.

In this application, Galmed Pharmaceuticals describes a selection of novel salts of aramchol which enhances the solubility and absorption thereof, and therefore, the bioavailability thereof. Amine salts of aramchol, in particular the salts of meglumine, lysine and tromethamine, were selected after a permeability study on rats. However, the present application does not disclose any findings relative to the use of amine salts of aramchol in the treatment of NAFLD and NASH.

The application US2015/0374794A of Novo Nordisk refers to a glucagon derivative used in the treatment of diabetes and fatty liver disease. Novo Nordisk obtained results in NASH regression with the corresponding medicinal product, Victoza®. The findings are published in a study of 52 patients, but the research was conducted with the former definition of NASH reversal parameters, which accepts steatosis suppression as a criterion.

Intercept Pharmaceuticals, with its medicinal product OCA (obeticholic acid), a farnesoid X receptor (FXR) selection analogue and agonist, demonstrated an action on steatosis, inflammation and fibrosis in a phase IIb study “FLINT” (conducted on 283 patients), without arriving at a significant result in NASH reversal.

Moreover, obeticholic acid (OCA) was not successful in confirming effects on NASH and fibrosis in the subsequent phase IIb study in Japan, which discredited the efficacy of the molecule in the treatment of NASH and fibrosis. Moreover, obeticholic acid showed numerous side-effects, including significant pruritus and in particular increased cholesterol, which is not acceptable for patients presenting with a cardiovascular risk.

Further pharmaceutical firms have a different approach and target inflammation and prolapse. The U.S. Pat. No. 9,115,073B2 discloses a molecule developed by Genfit, which exhibits intrinsic agonist properties of peroxisome proliferator-activated receptors (see detail hereinafter). These receptors are proteins of the superfamily of nuclear receptors naturally binding lipids and acting as a transcription factor of the target genes involved particularly in the metabolism and adipogenesis. The agonist properties of this molecule make it a candidate for the treatment of metabolic disorders and/or inflammatory diseases, and in particular peripheral and central diseases associated with metabolic syndrome, including non-alcoholic steatohepatitis.

This patent has given rise to various studies, particularly one clinical study on NASH patients. NASH severity is assessed according to histological criteria resulting in an NAS index/score. The higher the NAS index/score, the more severe the histological expression of NASH. During this study, the causal relationship between the metabolic effects of the molecule and the histological response was not assessed. Furthermore, few differences were observed compared to the placebo in the groups of patients having an NAS index/score less than 4.

Further experimental medicinal products merely target an advanced stage of fibrosis and cirrhosis. Gilead Sciences is studying two molecules (simtuzumab and GS-4997) for the treatment of NASH and of primary sclerosing cholangitis (PSC). Simtuzumab is an anti-LOXL2 monoclonal antibody tested within the scope of a phase Ilb study, but for which the findings are not yet known. GS-4997, for its part, an ASK1 (apoptosis signal-regulating kinase) inhibitor is described in the U.S. Pat. No. 8,440,665B2 and U.S. Pat. No. 8,927,582 B2. The molecule is currently under evaluation in a phase II study of patients suffering from NASH and from moderate to severe liver fibrosis. Information is provided in the application US 2015/0342943 AI for the treatment of liver diseases.

The application WO2015143367 (Tobira Therapeutics) describes a novel type of medicinal product, cenicriviroc (or CVC), an HIV entry inhibitor which inhibits access to the CCRS receptor, as a candidate for the treatment of NASH in adult subjects suffering from liver fibrosis. The phase II study (“CENTAUR”) is in progress. Cenicriviroc is administered per os at 150 mg, daily in the morning with food.

The U.S. Pat. No. 8,658,787 B2 (Galectin Therapeutics) relates to a polysaccharide formulation containing galactose, particularly galacto-rhamnogalacturonate GR-MD-02. In the phase II study, GR-MD-02 is administered in doses of up to 8 mg/kg/week. The study is not scheduled to be completed before 2018. The application WO2015/175381 AI (Conatus Pharmaceuticals) treats chronic liver disease-related complications with caspase inhibitors, such as emricassan. Conatus Pharmaceuticals states that apoptosis and caspase activity are correlated with the stage of cirrhosis. A clinical study is ongoing on patients suffering from liver cirrhosis in order to determine whether 25 mg doses of emricassan could have a clinical benefit by reducing the apoptosis rate in cirrhotic patients, and thus potentially reduce disease progression as determined by biomarkers.

The patent application WO2013/169648A relates to a pharmaceutical composition combining a plurality of active ingredients, particularly a DGAT1 inhibitor, in combination with a medicinal product lowering the blood triglyceride level, and one or a plurality of excipients. This application cites the use of fenofibric acid in combination, as a molecule making it possible to lower the triglyceride level. Furthermore, use within the context of NAFLD treatment is cited. The active ingredients are used in combination, so as to target a maximum number of physiological mechanisms aiming to lower the blood triglyceride level, resulting in a composition that is difficult to use, the production whereof is complex and costly. Finally, DGAT1 catalyses triglyceride synthesis, the inhibition thereof therefore lowers triglyceride synthesis, the DGAT1 inhibitor is associated with a medicinal product lowering the blood triglyceride level. NAFLD corresponds to a build-up of fatty acid in the liver. Hypertriglyceridaemia is merely a phenomenon associated with the disease and a lowering of triglycerides in the blood is not equated to recovery from the disease.

The patent application EP2296659 relates to the treatment of obesity, type 2 diabetes, heart diseases and cancer. It relates to a combination of a DGAT1 inhibitor and a PPAR-α agonist. This application cites the use of fenofibric acid in combination, and the treatment of NASH and NAFLD. However, no data, nor any findings relative to the use of the composition of the context of NASH are provided. The patent application merely aims to lower the blood triglyceride level and treat dyslipidaemia, which are merely peripheral phenomena to the majority of liver diseases (particularly obesity) and does not relate to a method for treatment of liver diseases, such as NAFLD or NASH.

In sum, the latter two patent applications relate to lowering triglycerides in the blood and controlling dyslipidaemia which are peripheral parameters to liver diseases, but provides no solution for liver cell lesions.

A conclusion on these candidate treatments makes it possible to highlight that the main drawbacks of these experimental medicinal products lie in that they only act upon the consequences of inflammation, and not upon the causes thereof. Once the treatment is discontinued, the liver diseases progress further. Targeting all the effects of inflammation gives rise to a burdensome treatment for patients, and there is a need for a simple treatment, suitable for treatment of liver diseases and particularly NASH and all of the consequences associated with this condition. The treatment should if possible be easy to administer, have an advantageously lower production cost. Furthermore, all the existing medicinal products are merely at the clinical development stage and the effects thereof are debatable at the present time. There is therefore the need for a composition exhibiting satisfactory results in the treatment of NASH, and covering all the forms of expression of this disease, from the least severe to the most severe.

Consequently, there is currently an unmet need for medicinal products that are sufficient safe and effective, easy to administer, economical in the production thereof, to treat or forestall patients suffering from liver diseases, such as non-alcoholic fatty liver disease, non-alcoholic steatohepatitis, liver fibrosis or cirrhosis.

However, whereas most of the research focuses on combinations of molecules targeting a maximum number of peripheral physiological phenomena to liver diseases, the applicant discovered surprisingly that the use of fenofibric acid alone has interesting potential in the prevention and treatment of non-alcoholic fatty liver disease NAFLD, non-alcoholic steatohepatitis NASH, liver fibrosis and cirrhosis.

Fenofibric acid belongs to the family of fibric acids which are amphiphilic carboxylic acids characterised by the presence of a terminal carboxyl group (COOH), with polar and non-polar properties.

Originally, most fibrates are manufactured and marketed essentially in the form of esters, such as: clofibrate, fenofibrate. Fibrates have been used for 50 years in the treatment of numerous forms of hypercholesterolaemia, in association or not with statins. Although they are less effective at lowering the LDL cholesterol (bad cholesterol) and triglyceride level, the ability of fibrates to increase the HDL cholesterol (good cholesterol) level and lower the triglyceride level appears to lower insulin resistance when dyslipidaemia is associated with other characteristics of metabolic syndrome (hypertension and type 2 diabetes). Consequently, they are used in numerous cases of hyperlipidaemia. Fibrates are not suitable for patients presenting with low HDL levels.

It has been known for several years that fibrates induce peroxisome proliferation in rodents. This process is linked with the induction of the transcription of genes involved in peroximal β-oxidation and is mediated by specific transcription factors, hence referred to as peroxisome proliferator-activated receptors (PPAR).

Peroxisome proliferator-activated receptors are members of the superfamily of nuclear hormone receptors, which are transcription factors transmitting signals originating from soluble lipid factors (for example hormones, vitamins and fatty acids) in the genome. Nuclear receptors recognise and bind with DNA at the level of the response element (REs) and activate or repress the expression of a target gene.

Peroxisome proliferator-activated receptors are heterodimerised with the retinoid X receptor (RXR) and bind with the response elements, referred to as peroxisome proliferator response elements (PPRE).

At the present time, there are 3 different known peroxisome proliferator-activated receptor genes: α, δ (also known as β, NUC I, or FAAR), and γ.

Peroxisome proliferator-activated receptors exhibit distinct expression profiles, which suggests significant functional differences. PPARα is essentially expressed in tissues metabolising large quantities of fatty acids, such as the liver, kidneys, heart and muscle. PPAR-γ expression is elevated in adipose tissue, where it triggers adipocyte differentiation and induces the expression of critical genes for adipogenesis.

PPAR-α is a nuclear transcription factor (NTF), regulating potentially hundreds of genes involved in energy expenditure: lipid and lipoprotein synthesis and catabolism, fatty acid regulation vascular wall biology.

The natural ligands for PPAR-α are various fatty acids and derivatives such as 8(S) hydroxyeicosatetraenoic acid, 8(S) hydroxyeicosapentaenoic acid and leukotrienes B4, whereas fibrates are synthetic PPAR-α ligands. Some carboxylic acids have the ability to stimulate PPARs (α, γ and δ).

Fibrates, however, have not demonstrated to date properties on SPPARM selective receptors, identified more recently and which exhibit inter alia greater clinical benefits, particularly with less side-effects, as observed on the molecule K877 under development by Kowa Pharmaceutical (“Selective peroxisome proliferator-activated receptora modulators (SPPARMα): the next generation of peroxisome proliferator-activated receptor α-agonists” Fruchart; Cardiovascular Diabetology 2013, 12:82). On the other hand, the ability of fibrates to inhibit the platelet function is recognised, thus reducing cardiovascular and thrombotic risks (Arterioscler Thromb Vasc Biol-2009-Ali-706-11).

Fibrates may also influence other nuclear transcription factors such as LXR liver receptors and ANGPTLs which also play a key role in lipid biology. The process is extremely complex and very tissue-specific; this is also dependent on the presence or absence of a plurality of other proteins which serve as nuclear transcription factor co-activators or co-repressors. For example, in the liver and not in macrophages, fenofibrate is an LXR antagonist (important in triglyceride synthesis inhibition), whereas fenofibric acid (and not the ester form) does not have hepatic LXR antagonist capability in triglyceride synthesis but has on the other hand an LXR agonist activity to regulate the ABCA1 gene belonging to the ABC transporter family and involved in cholesterol transport.

Therefore, there are differences in respect of the intrinsic agonist activities of fibrates depending on whether the acid or ester is involved. By way of example, esters such as fenofibrate are merely agonists of PPARα and not of the 2 other isoforms, whereas bezafibrate which is an acid is a pan-agonist of the 3 PPAR α, β/δ and γ isoforms (Grygiel-Gorniak Nutrition Journal 2014, 13:17). The pharmacodynamic roles are therefore recognised as being different depending on whether there is esterification or amidation of the carboxyl group of the fibrate molecule (Mol. and Cell. Bioch. November 2010, 344, 91-98).

The fibrates most tested in clinical trials and most prescribed to date are gemfibrozil, bezafibrate and fenofibrate. They are marketed under different trade names.

The applicant discovered fortuitously that the use of fenofibric alone has interesting potential in the prevention of liver diseases, particularly NAFLD, NASH, liver cirrhosis and liver fibrosis, but also that, unlike fenofibrate, fenofibric acid offers the advantage of not requiring liver metabolism to modify the structure thereof and exercise a PPARα agonist activity.

Indeed, fenofibric acid, or one of the pharmaceutically acceptable salts thereof, is theoretically safer than fenofibrate as it does not require liver activation.

Finally, the applicant discovered that the use of fenofibric acid alone plays an agonist role on PPARα, and by means of the pleiotropic activities thereof, suffices to play an important beneficial role in the peripheral cardio-metabolic conditions making up metabolic syndrome:
Obesity,
Insulin resistance,
Type II diabetes,
Cholesterolaemia,
Hypertriglyceridaemia,
Hypertension,
Hyperuricaemia (gout),
Platelet coagulation/aggregation,
Systemic inflammation,

of which NASH is the expression in the liver. Indeed, fenofibric acid which is a selective PPARα receptor activator is as such the only active ingredient that covers almost all of the conditions making up NASH.

### DESCRIPTION OF THE INVENTION

It is reiterated firstly that the invention relates to a composition comprising by way of sole active ingredient fenofibric acid or one of the pharmaceutically acceptable salts thereof for use in the treatment of liver diseases.

Advantageously, the pharmaceutically acceptable salt of fenofibric acid consists of a choline salt.

The choline salt enhances the aqueous solubility of fenofibric acid, enabling in the case of oral administration easier uptake, and in the case of intravenous administration the obtaining of an easier-to-inject solution.

Advantageously, the pharmaceutically acceptable salt of fenofibric acid is the metformin salt.

The metformin salt enhances the efficacy of fenofibric acid.

Surprisingly, it was discovered that fenofibric acid in salt form with metformin, which is a novel molecular entity, provides different advantageous effects from the sum of fenofibric acid and metformin taken individually. Indeed, the metformin salt enables a synergistic action of the active ingredient particularly influencing efficacy in the treatment of liver diseases and particularly of non-alcoholic steatohepatitis or non-alcoholic fatty liver disease associated with metabolic syndrome diseases such as diabetes and obesity.

Advantageously, the liver disease consists of non-alcoholic steatohepatitis.

Advantageously, the liver disease consists of non-alcoholic fatty liver disease.

Advantageously, the liver disease consists of liver fibrosis.

Advantageously, the liver disease consists of cirrhosis.

Advantageously, the mode of administration consists of administration by the sublingual route.

Advantageously, the mode of administration consists of administration per os.

Advantageously, the mode of administration consists of administration by the subcutaneous route.

Advantageously, the mode of administration consists of administration by the intravenous route.

The modes of administration by the sublingual, subcutaneous and intravenous routes make it possible to reduce the first-pass effect.

Advantageously, the pharmaceutical form consists of a ring.

The ring form offers the advantage of having a greater contact area.

Advantageously, the pharmaceutical form consists of the sublingual ring, sublingual stick, buccoadhesive ring, buccoadhesive stick, freeze-dried ring, soluble ring, orally dissolving ring, effervescent ring form or of a single-dose oral solution.

Advantageously, the distribution profile of fenofibric acid includes fenofibric acid particles, at least 50% of the particles being less than 2000 nm in size and all of the particles being less than 5000 nm.

Micronisation of the fenofibric acid enhances the solubility thereof and the penetration thereof through the mucous membranes.

Advantageously, the pharmaceutical form consists of a sublingual ring including fenofibric acid particles, at least 50% of the particles being less than 2000 nm in size and all of the particles being less than 5000 nm.

Advantageously, the unit dose of fenofibric acid administered is between 10 and 110 mg.

Advantageously, the unit dose of fenofibric acid is between 10 and 50 mg.

Advantageously, the daily dose of fenofibric acid is between 25 and 110 mg.

Advantageously, the composition includes at least one excipient, particularly at least one among binders, disintegrating agents, diluents, lubricants, surfactants, buccoadhesive agents, uptake activators/promoters, buffer agents, flow agents, colorants, flavours, sweeteners, solvents or preservatives.

According to a further aspect, the invention relates to a method for preparing a composition including by way of active ingredient fenofibric acid characterised in that this active ingredient is mixed with excipients, particularly binding agents, by the wet granulation process. The grains formed are then dried and calibrated, then mixed with the remaining excipients of the composition prior to compression of the powder mixture obtained so as to exhibit the form sought (for example, ring, stick).

The invention relates to a use of a composition comprising by way of sole active ingredient fenofibric acid or one of the pharmaceutically acceptable salts thereof for obtaining a medicinal product intended for use in the treatment of liver diseases, particularly NAFLD, NASH, liver fibrosis, cirrhosis.

The invention relates to a method for the treatment liver diseases, particularly NAFLD, NASH, liver fibrosis, cirrhosis comprising the use of fenofibric acid by way of sole active ingredient.

### BRIEF DESCRIPTION OF THE FIGURES

FIG. 1 is a solubility curve as a function of pH.

### DETAILED DESCRIPTION

The present invention relates to the use of fenofibric acid, or one of the pharmaceutical acceptable salts thereof, or one of the polymorphous crystalline forms thereof, in the preparation of a medicinal product for the treatment and prevention of liver diseases, in particular non-alcoholic steatohepatitis (NASH), non-alcoholic fatty liver disease (NAFLD), liver fibrosis, and cirrhosis.

The invention also relates to the use of 2-(4-(4-chlorobenzoyl) phenoxy)-2-methylpropanoic (C17H15ClO4) (hereinafter), among one of the pharmaceutically acceptable salts thereof or one of the polymorphous crystalline forms thereof, suitable for use in a pharmaceutical composition to prevent or treat liver disease.

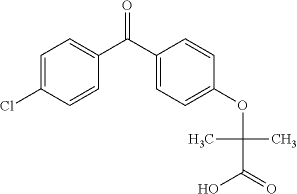 [SMILES: CC(C)(Oc1ccc(C(=O)c2ccc(Cl)cc2)cc1)C(=O)O]

The term “pharmaceutically acceptable salt” denotes, by way of example and non-exhaustively, the salts of: ethanolamine, meglumine, L-lysine, tromethamine, arginine, ornithine, choline and metformin.

The term “polymorphous crystalline form” denotes by way of example and non-exhaustively: the polymorph I form (melting point at 175° C.), the polymorph II form of fenofibric acid prepared particularly from a suspension of fenofibrate in isopropanol (melting point of about 184° C.).

The pharmaceutical composition according to the invention may be administered per os, via the mucous membranes, by the parenteral or topical route. According to one mode of administration, the composition is administered by the enteral route, such as, for example, a sublingual ring, a sublingual scored stick, a buccoadhesive ring or a buccoadhesive stick, a soluble ring, a freeze-dried ring, an effervescent ring, a fast orally disintegrating ring or a single-dose solution.

According to a preferred mode of administration, the composition is administered via the oral mucous membrane. There are three different drug delivery categories in the oral cavity: sublingual, oral and local/localised administration. The sublingual mucous membrane is relatively permeable, providing rapid uptake and acceptable bioavailabilities for numerous active molecules; it is convenient, accessible, and generally well accepted (Drug delivery via the mucous membranes of the oral cavity, J. Pharm Sci, 81: . . . 1-10, 1992). The oral mucous membrane is much less permeable than the sublingual region, but offers the same advantages, including in respect of liver bypass: no liver first-pass effect, enzyme breakdown in the gastrointestinal tract region.

A further feature of the environment of the oral cavity is the presence of saliva produced by the saliva glands. Saliva is an aqueous fluid containing 1% organic and inorganic substances. Salivary pH varies from 5.5 to 7 depending on the saliva flow rate. The daily volume of saliva is between 0.5 and 2 litres depending on the individuals and certain physiological factors (stress, etc.). The mean volume, available to hydrate and disintegrate pharmaceutical forms present in the oral mucous membrane is about 10 ml.

Local administration to oral cavity tissues has a certain number of applications, including the treatment of toothache, periodontal diseases, bacterial and fungal infections, mouth ulcers and other dental problems. However, this type of administration has not been tested on hypolipaemic medicinal products such as fibrates while the first-pass effect has been reported for fenofibrate (Bays H E, et al J Clin Lipidol 2008; 2: 426-435), and therefore on the corresponding fibric acids, insoluble in water, but for which solubility increases as a function of pH in buffered media.

The formulations intended to be administered via the oral mucous membrane contain uptake promoting agents and/or buffer agents, and/or surfactants well-known to those skilled in the art in order to optimise the oral uptake of medicinal products. A non-exhaustive list is provided herein: 23-lauryl ether, aprotinin, azone, cyclodextrin, dextran sulphate, lauric acid, lauric acid and propylene glycol, lysophosphatidylcholine, menthol, methoxysalicylate, methyl oleate, oleic acid, phosphatidylcholine, polyoxyethylene, polysorbate 80, sodium EDTA, sodium glycocholate, sodium glycodeoxycholate, sodium lauryl sulphate, sodium salicylate, sodium taurocholate, sodium taurodeoxycholate, etc.

The formulations intended to be administered via the oral mucous membrane contain micronised fenofibric acid so as to enhance the solubility thereof in saliva and the penetration thereof through the oral mucous membrane (via paracellular and transcellular routes). The particle size distribution of the micronised fenofibric acid is characterised in that more than 50% of the particles are less than or equal to 2000 nm, and all the particles are less than 5000 nm in size.

The particles are measured using a Malvern type laser granulometer or equivalent, and a wet process-validated method (wetting with a surfactant) is preferred.

One of the preferred pharmaceutical compositions of the invention is a sublingual micronised fenofibric acid ring, where the ring is kept in the oral cavity, under the tongue, for the time required to dissolve and absorb the medicinal product.

The following examples are given to illustrate the invention and in no way represent a restriction thereof.

### EXAMPLES

### Example 1: In Vitro Cellular Model in NASH/NAFLD

The applicant decided to explore in vitro approaches to test fenofibric acid as a medicinal product inhibiting progression of non-alcoholic fatty liver disease/steatohepatitis. In this field, only a few data obtained in NASH/NAFLD using cellular models have been published, whereas for other liver diseases, the research models and means are more numerous and more studied. In general, immortalised cell lines and primary cell cultures are widely used to develop in vitro models for research. The immortalised primary cells are candidate cells selected for the in vitro study due to the stable phenotype thereof, but also due to a culture method that is simple and standardised to carry out.

Experimental Protocol

The immortalised human cells were cultured in an enriched medium containing increasing fenofibric acid concentrations. The purpose of the study consisted of measuring the intracellular lipid droplet content. The entire staining procedure is conducted at ambient temperature while protecting the samples from direct light. The images were acquired with a fluorescence microscope.

Results

The intracellular lipid droplet content was determined by labelling. Exposing the cells to fenofibric acid concentrations μM for 24 hours induces a dose-dependent fat reduction. The microscopic images show the presence of a significantly lesser quantity and size of cytoplasmic lipid droplets. This trend is all the more noteworthy at the highest fenofibric acid concentration wherein the quantity and size of the droplets decreased in relation to that observed with the lowest dose.

### Example 2: In Vivo Mouse Model of Non-Alcoholic Steatohepatitis

During an experiment conducted for the invention, it was demonstrated that fenofibric acid displayed a pharmacological activity when administered per os in a validated mouse model of NASH. The applicant chose a mouse model used and validated for non-alcoholic steatohepatitis (NASH) and hepatocellular carcinoma (HCC) as it is particularly suitable for studying the efficacy of the candidate medicinal product which has an LXR agonist activity, unlike fenofibrate (ester).

Experimental Protocol

Fenofibric acid is studied at 2 doses and administered per os once daily. The positive control is telmisartan and fenofibrate (ester) is selected as the negative control, more specifically as comparative data (as per the respective reference articles Liver Int. 2009 August; 29(7):988-96 and Eur J Pharmacol. 2016 Feb. 5; 772:22-32).

The number of male mice is 60 for the curative part of the study. They follow the normal light cycle, and are divided into enriched ventilated cages, after a one-week acclimatisation period.

The mice are fed with high-fat content feed and fructose-enriched tap water. The mice are treated with the references or with the test formulation according to 2 doses, with daily administration per os for about 2 months.

The mice not exhibiting sufficient weight gain or conforming biochemical parameters (ALT and AST) are withdrawn from the remainder of the study and the mice incorporated in the curative part of the study are randomised. At different weeks of treatment, blood is collected to measure the blood glucose level, insulin, total cholesterol and triglyceride level, as well as the ALT and AST levels. After the final collection, the mice are sacrificed to measure the total hepatic cholesterol, triglycerides and fatty acid levels.

Liver samples are also collected for histological analysis and the NAS score/index.

Results

The macroscopic and microscopic examination of the liver showed that oral administration of fenofibric acid prevented the development of fatty liver disease. Fenofibric acid also notably reduced the plasma ALT concentration, which is a marker of liver dysfunction.

In the light of the results, the applicant observed that fenofibric acid could significantly inhibit the increase in the weight of the liver and the triglyceride level. Moreover, among the biochemical parameters, the ALT levels decreased substantially.

Furthermore, we observed that fenofibric acid could significantly inhibit the increase in the fatty liver disease activity score and the fibrotic zones.

### Example 3: In Vitro Oral Penetration Study

One of the preferred modes of administration is the oral mucous membrane route. In order to achieve this, the applicant prepared fenofibric acid solutions from samples provided by the supplier Harman Finochem (India). The in vitro penetration of the fenofibric acid formulation is evaluated using Franz type diffusion cells at 37° C., a system routinely used to evaluate the in vitro penetration of compounds through the skin, but with the oral mucous membrane as the membrane in this instance.

Experimental Protocol

Membrane Preparation:

The applicant chose pig oral tissue because it has a non-keratinised morphology, relatively similar to the human oral epithelium (Gandhi, R. B. and Robinson. Adv Drug Deli Rev, 13:43-74, 1994). The oral mucous membranes are separated by removing the underlying connective tissue using surgical scissors, to ensure that the basement membrane is still present. The tissue sections therefore have a thickness of at least 500 μm and an approximate surface area of about 4 to 5 cm2. Each membrane used is mounted between the donor and receptor compartments of the Franz cell system.

System Preparation:

The receptor compartment is filled with pH 7.4 buffer at 37° C. and the donor compartment with micronised fenofibric acid solution at 37° C. of different concentrations (from 0.2 mg/ml) with a pH also adjusted to pH 7.4 or a solution of fenofibric acid (0.5 mg/ml) in buffer solutions of different pH (4.5, 6.0, 6.8 and 7.4). Samples are taken from the receptor compartment at a predetermined time interval (2, 5, 10, 15, 30, 60, 90, 120, 150 and 180 minutes), replaced by the same volume of buffer and subsequently analysed using the HPLC method.

The quantity of fenofibric acid presented in the donor compartment is determined as a function of time. The permeability coefficient (P) may be calculated based on the linear part of the measurements made on the first times (regression).

The transport pathway of fenofibric acid is theoretically the transcellular pathway due to the lipophilic nature thereof. Lipophilicity is increased at elevated pH, which leads in theory to a significant increase in penetration through the porcine oral mucous membrane as a function of pH.

Yield and Extraction of the Active Substance Present in the Membrane:

The mucous membrane used in the test is detached from the diffusion cell after the final sampling. It is disaggregated using a mortar and pestle and the contents are extracted with an alcoholic solution and passed in a sonicator bath and cooled if necessary. The organic layer is centrifuged to separate the cell components. The extract is diluted if required prior to being analysed by HPLC to determine the overall yield (Donor compartment quantity=Receptor compartment quantity+quantity extracted from membrane).

Results:

The solubility of fenofibric acid increases as a function of pH and it is greatest at the pH values closest to neutrality. The results show a noteworthy penetration of fenofibric acid through the porcine oral mucous membrane, from the first samples, with more rapid kinetics at higher concentrations and at elevated pH levels. The yield of the study is close to 100%, accounting for the analytical margin of error, relative to the initial theoretical concentration, which makes it possible to validate the test.

In conclusion, fenofibric acid is rapidly absorbed via the oral mucous membrane under the different conditions tested. The molecule is a good candidate for a formulation with uptake in the oral cavity.

### Example 4: Solubility Test of Different Salts of Fenofibric Acid

The purpose of this example is to determine the solubility characteristics of different crystalline forms and salts of fenofibric acid, with a view to administration by the oral mucosal route, in order to treat or forestall patients of liver diseases, such as fatty liver disease.

The solubilisation kinetics are determined by placing fenofibric acid samples at an excess in an aqueous buffered solution at pH 6.0, equivalent to salivary pH. Samples are taken after 0.5, 1, 2, 4, 6, 12 and 24 hours. They are analysed by HPLC after centrifugation and filtration of the samples on a 0.45 μm PTFE filter.

The samples are received from several Indian and Chinese suppliers. Fenofibric acid form II is obtained from a suspension of fenofibrate (ester) in isopropanol.

The results in terms of solubility at saturation (after 24 hours of stirring) are given in the table below and are expressed as per the criteria of the European Pharmacopoeia in force at the time of the tests. The melting points are obtained by DSC (endothermic peak, onset measurement).

| | Molecular | Melting point in | Solubility at |
|---|---|---|---|
| Substances | weight | ° C. | saturation |
| Micronised fenofibric | 318.75 | 175 | Slightly soluble |
| acid | | | |
| Salt of ethanolamine | 379.83 | 121 | Sparingly soluble |
| Salt of meglumine | 513.96 | 114 | Sparingly soluble |
| Salt of L-lysine | 464.94 | 163 | Soluble |
| Salt of tromethamine | 439.89 | 198 | Slightly soluble |
| Salt of choline | 421.91 | 209 | Very soluble |
| Fenofibric acid form II | 318.75 | 184 | Slightly soluble |

Note:
as a reminder, fenofibrate has a melting point of 80° C.

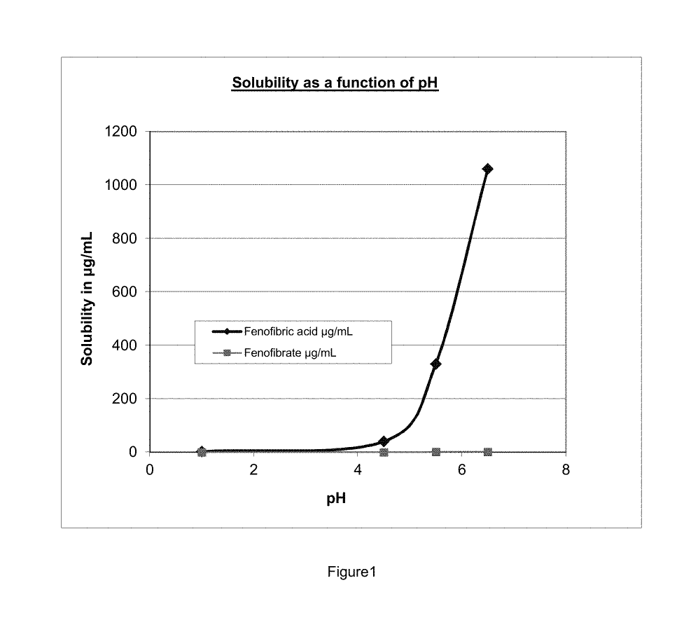

The results were completed by an aqueous phase solubility study of fenofibric acid as a function of pH, alone or associated in a 50:50 binary mixture with a surfactant, and compared to fenofibrate. The table below summarises the results and shows superior solubility at the pH values closest to neutrality. (See FIG. 1)

| | Fenofibric Acid | Micronised fenofibrate |
|---|---|---|
| Aqueous medium | Solubility (μg/mL) | Solubility (μg/mL) |
| Purified water | 20 | ~0 |
| 0.01M Sodium lauryl | 90 | 70 |
| sulphate | | |
| 0.1M Sodium lauryl | 810 | 1310 |
| sulphate | | |
| 0.1N HCl | 2 | ~0 |
| Phosphate buffer | 40 | ~0 |
| pH = 4.5 | | |
| Phosphate buffer | 330 | 1 |
| pH = 5.5 | | |
| Phosphate buffer | 1060 | 1 |
| pH = 6.5 | | |
| | Fenofibric Acid | |
| | Solubility | Micronised fenofibrate |
| Surfactant | in water (mg/g) | Solubility in water (mg/g) |
| Labrasol | 55 | 4.6 |
| Tween 80 | 61 | >71 |
| Cremophor EL | 56 | ~50 |
| Solutol HS15 | 60 | >100 |
| Cremophor | 59 | ~25 |
| RH40 | | |

### Example 5: Dissolution Kinetics of Fenofibric Acid

The applicant studied the dissolution profile of several batches of fenofibric acid received from several suppliers. The starting material batches include different granulometric characteristics including one standard grade (non-micronised) and several micronised batches.

The batches of fenofibric acid were prepared in various pharmaceutical forms including the following excipients: hypromellose, milk protein concentrate, corn starch, Lactose monohydrate (0.4 mg/50 mg of active ingredient), sodium lauryl sulphate as surfactant, magnesium stearate, and talc as a flow agent required during production.

| | | | Specific | |
|---|---|---|---|---|
| | | | exchange | |
| Pharmaceutical | | | surface area | Active ingredient |
| forms | Dosages | | mm²/ml | grain size |
| Round tablet | 50/100 | mg | + | Standard/micronised |
| Ring (or POLO | 10/50/100 | mg | +++ | Micronised |
| type tablet) | | | | |
| Scored stick | 25/50/75/100 | mg | ++ | Micronised |

Dissolution tests were carried out on most of the formulation in media such as acetate buffer at pH 6.0, a phosphate buffer at pH 4.5 and purified water. Similarly, the rings were tested in gastric media simulating fed and fasting conditions, with addition of enzymes (media referred respectively as FeSSIF and FaSSIF) were also tested after the 50 and 100 mg doses. The USP type II paddle system was retained for the tests with 1000 ml of volume, 37° C. and the samples, taken at 0, 2, 5, 10, 15, 20, 30, 45 and 60 minutes, were analysed by HPLC without replenishing the media.

Results

The results show that micronised fenofibric acid alone is totally dissolved after 60 minutes and the dissolution kinetics are more rapid for the most micronised batch (100% of particles less than 5000 nm and >53% of particles less than 2000 nm).

For the pharmaceutical formulations, the dissolution kinetics are slowest for the “tablet” form which exhibits the smallest specific exchange surface area.

At pH ≥6.0, over 80% of the fenofibric acid is dissolved after 10 minutes on the ring and stick forms, regardless of the dosages tested.

Moreover, in the media simulating fasting and fed conditions, the dissolution kinetics remain equally rapid. In the FaSSIF and FeSSIF media, over 80% of the fenofibric acid is dissolved after 10 minutes and the entirety after 30 minutes. There are no differences between the two media.

In conclusion, the impact of the grain size of the fenofibric acid on the dissolution properties thereof was demonstrated, with superior dissolution for the most micronised form. Moreover, the results demonstrate that the dissolution is rapid and dependent on the specific exchange surface area, that there is no difference associated with feed intake as the results obtained for the FaSSIF and FeSSIF media are similar in the pharmaceutical forms studied.

1. A composition comprising as of sole active ingredient fenofibric acid or one of the pharmaceutically acceptable salts thereof for use in the treatment of liver diseases.

2. The composition for use in the treatment of liver diseases according to claim 1 wherein the pharmaceutically acceptable salt of fenofibric acid consists of a choline salt.

3. The composition for use in the treatment of liver diseases according to claim 1 wherein the pharmaceutically acceptable salt of fenofibric acid consists of a metformin salt.

4. The composition according to claim 1 for use in the treatment of liver diseases associated with obesity and diabetes.

5. The composition for use in the treatment of liver diseases according to claim 1 wherein the liver disease consists of non-alcoholic steatohepatitis.

6. The composition for use in the treatment of liver diseases according to claim 1 wherein the liver disease consists of non-alcoholic fatty liver disease.

7. The composition for use in the treatment of liver diseases according to claim 1 wherein the liver disease consists of liver fibrosis.

8. The composition for use in the treatment of liver diseases according to claim 1 wherein the liver disease consists of cirrhosis.

9. The composition for use in the treatment of liver diseases according to claim 1 wherein it is in a form suitable for administration by the sublingual route.

10. The composition for use in the treatment of liver diseases according to claim 1 wherein it is in a form suitable for administration by the enteral route.

11. The composition for use in the treatment of liver diseases according to claim 1 wherein it is in a form suitable for administration by the subcutaneous route.

12. The composition for use in the treatment of liver diseases according to claim 1 wherein it is in a form suitable for administration by the intravenous route.

13. The composition for use in the treatment of liver diseases according to claim 1 wherein the pharmaceutical form consists of a ring.

14. The composition for use in the treatment of liver diseases according to claim 1 wherein the pharmaceutical form consists of the sublingual ring, sublingual stick, buccoadhesive ring, buccoadhesive stick, freeze-dried ring, soluble ring, orally dissolving ring, effervescent ring form or of a single-dose oral solution.

15. The composition for use in the treatment of liver diseases according to claim 13 wherein the distribution profile of fenofibric acid includes fenofibric acid particles, at least 50% of the particles being less than 2000 nm in size and all of the particles being less than 5000 nm.

16. The composition for use in the treatment of liver diseases according to claim 9 wherein the pharmaceutical form consists of a sublingual ring including fenofibric acid particles, at least 50% of the particles being less than 2000 nm in size and all of the particles being less than 5000 nm.

17. The composition for use in the treatment of liver diseases according to claim 1 wherein a unit dose of fenofibric acid is between 10 and 110 mg.

18. The composition for use in the treatment of liver diseases according to claim 1 wherein a unit dose of fenofibric acid is between 10 and 50 mg.

19. Composition for use in the treatment of liver diseases according to claim 1 wherein the composition includes at least one excipient, particularly at least one among binders, disintegrating agents, diluents, lubricants, surfactants, buccoadhesive agents, uptake activators/promoters, buffer agents, flow agents, colorants, flavours, sweeteners, solvents or preservatives.
